(19) United States
(12) Patent Application Publication
(10) Pub. No.: US 2026/0275263 A1
(43) Pub. Date: Sep. 17, 2026

(54) ADDING DEVICE AND ADDING METHOD OF PRODUCED SUBSTANCE BY CULTURED ORGANISM

(71) Applicant: NTT, Inc., Tokyo (JP)

(72) Inventors: Sosuke IMAMURA, Musashino-shi (JP); Kazuhiro TAKAYA, Musashino-shi (JP)

(73) Assignee: NTT, Inc., Tokyo (JP)

(21) Appl. No.: 19/133,256

(22) PCT Filed: Dec. 2, 2022
(86) PCT No.: PCT/JP2022/044601

§ 371 Date: May 28, 2025

(51) Int. Cl. C12M 1/00; C12M 1/12; C12M 1/34

(52) U.S. Cl. CPC C12M 29/04; C12M 23/06; C12M 41/32

(57) ABSTRACT

In an embodiment, an adding device of a produced substance by a cultured organism includes a culture tube and a semipermeable membrane section. A culture solution containing the cultured organism flows through a channel inside the culture tube, and the culture tube extends into a medium. The semipermeable membrane section is formed in a portion of the culture tube extending inside the medium. The semipermeable membrane section allows the substance produced by the cultured organism but not cells of the cultured organism to pass therethrough, thereby allowing the produced substance to permeate the medium from the channel.

### TECHNICAL FIELD

An embodiment of the present invention relates to an adding device and an adding method of a produced substance by a cultured organism.

### BACKGROUND ART

Substances produced by cultured organisms such as algae, fungi, or the like and added to media such as soil, water, or the like are used to promote growth of plants such as crops or the like. For example, a metabolite, protein (enzyme), or the like produced by a cultured organism is added to a medium to promote plant growth. In adding a produced substance to a medium, for example, a centrifuge or the like is used to separate a culture solution that contains a cultured organism into cells of the cultured organism and the produced substance, and then, the separated produced substance is added to the medium. In another example, the culture solution containing the cultured organism, that is a mixture of the cells and the produced substance, is added to the medium.

In adding a produced substance to a medium as described above, it is necessary to stop culturing a cultured organism before adding the produced substance to the medium. That is to say, the cultured organism is not cultured continuously. In adding a produced substance to a medium, there is a demand for adding the substance produced by a cultured organism to a medium without stopping culture of the cultured organism, that is, while continuing culture of the cultured organism.

### CITATION LIST

### Non Patent Literature

Non Patent Literature 1: Qu Y, Chen X, Ma B, Zhu H, Zheng X, Yu J, Wu Q, Li R, Wang Z, Xiao Y. Extracellular Metabolites of Heterotrophic Auxenochlorella protothecoides: A New Source of Bio-Stimulants for Higher Plants. Mar Drugs. 2022 Sep. 7; 20 (9): 569. doi: 10.3390/md20090569. PMID: 36135758; PMCID: PMC 9505405.

Non Patent Literature 2: Garcia-Gonzalez J, Sommerfeld M. Biofertilizer and biostimulant properties of the microalga Acutodesmus dimorphus. J Appl Phycol. 2016;28:1051-1061. doi: 10.1007/s10811-015-0625-2. Epub 2015 May 29. PMID: 27057088; PMCID: PMC4789255.

Non Patent Literature 3: Elsorra E. Idriss, Oliwia Makarewicz, Abdelazim Farouk, Kristin Rosner, Ralf Greiner, Helmut Bochow, Thomas Richter, Rainer Borriss. Extracellular phytase activity of Bacillus amyloliquefaciens FZB 45 contributes to its plant-growth-promoting effect. Microbiology (2002), 148, 2097-2109. doi: 10.1099/00221287-148-7-2097

### SUMMARY OF INVENTION

### Technical Problem

An object of the present invention is to provide an adding device and an adding method of a produced substance by a cultured organism, which enables the substance produced by the cultured organism to be added to a medium while continuing culture of the cultured organism.

### Solution to Problem

In an embodiment of the present invention, an adding device of a produced substance by a cultured organism includes a culture tube and a semipermeable membrane section. A culture solution containing the cultured organism flows through a channel inside the culture tube, and the culture tube extends into a medium. The semipermeable membrane section is formed in a portion of the culture tube extending inside the medium. The semipermeable membrane section allows the substance produced by the cultured organism but not cells of the cultured organism to pass therethrough, thereby allowing the produced substance to permeate the medium from the channel.

### Advantageous Effects of Invention

According to the present invention, it is possible to provide an adding device and an adding method of a produced substance by a cultured organism, which enables the substance produced by the cultured organism to be added to a medium while continuing culture of the cultured organism.

### BRIEF DESCRIPTION OF DRAWINGS

FIG. 1 is a schematic view illustrating an example of an adding device of a substance produced by a cultured organism according to an embodiment.

FIG. 2 is a schematic view illustrating a configuration of an extension (first extension) extending inside soil, that is a medium, in a culture tube of the adding device according to the embodiment.

### DESCRIPTION OF EMBODIMENTS

Hereinafter, an embodiment of the present invention will be described with reference to the drawings.

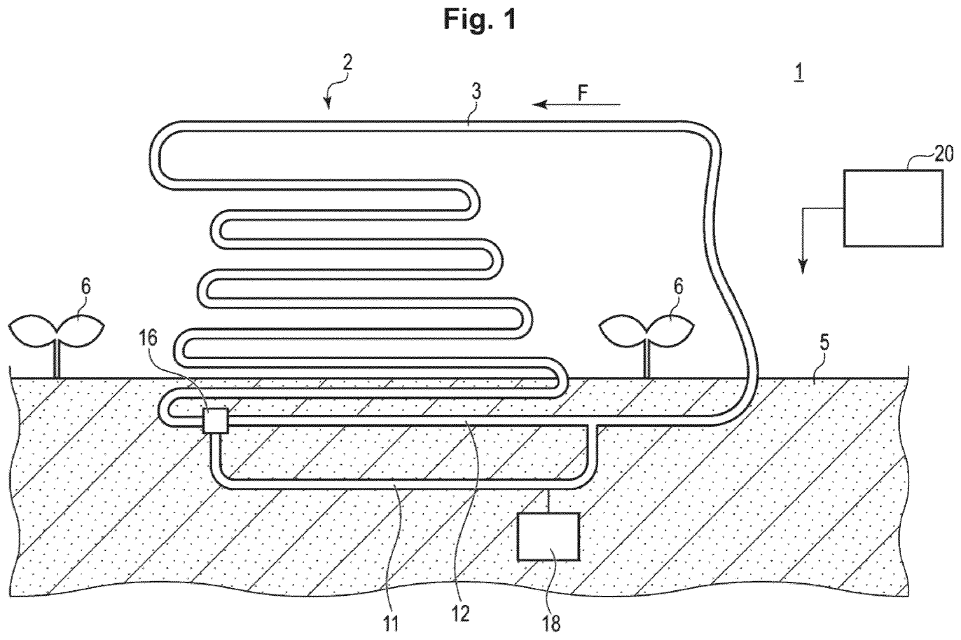

FIG. 1 illustrates an example of an adding device 1 of a substance produced by a cultured organism according to an embodiment. In the example illustrated in FIG. 1, the adding device 1 includes a culture tube 2. The culture tube 2 has an inner part provided with a channel 3 through which a culture solution containing the cultured organism flows. In the adding device 1, the cultured organism is cultured while the culture solution flows through the channel 3 inside the culture tube 2. In the example illustrated in FIG. 1, the culture solution flows through the channel 3 and circulates within the channel 3 (arrow F). Examples of the cultured organism to be cultured include algae and fungi such as Bacillus subtilis.Although one kind of cultured organism is basically cultured in the culture tube 2, a plurality of kinds of cultured organisms may be cultured in the culture tube 2.

The culture tube 2 extends into soil 5, that is a medium, and has a portion that extends inside the soil 5. A plant 6 such as a crop is planted in the soil 5. The medium into which the culture tube 2 extends is not limited to the soil 5 and may be water. In this case, the culture tube 2 extends into water, and in a place where the culture tube 2 is extended, a plant is grown by, for example, hydroponic cultivation.

In the example illustrated in FIG. 1, the culture tube 2 includes an extension (first extension) 11 extending inside the soil 5, that is the medium, and an extension (second extension) 12 extending in parallel with the extension 11. In other words, the culture tube 2 has the extensions 11 and 12 arranged in parallel. In the example illustrated in FIG. 1, the extension 12 extends inside the soil 5, but in another example, the extension 12 may extend outside the soil 5, that is the medium. Even in this case, it should be noted that the extension 11 extends inside the soil 5, and the extension 12 extends in parallel with the extension 11.

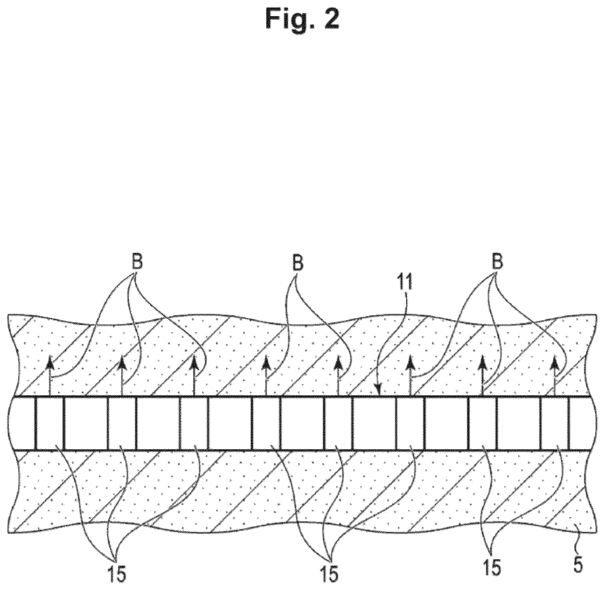

FIG. 2 illustrates an example of a configuration of the extension (first extension) 11 of the culture tube 2 extending inside the soil 5. As illustrated in FIG. 2 and the like, semipermeable membrane sections 15 are formed in the extension 11. In other words, the semipermeable membrane sections 15 are formed in a portion of the culture tube 2 extending inside the soil 5, that is the medium. In the example illustrated in FIG. 2, the extension 11 is provided with a plurality of semipermeable membrane sections 15, and the plurality of semipermeable membrane sections 15 is apart from one another at an interval. A semipermeable membrane in each semipermeable membrane section 15 forms the culture tube 2.

In each semipermeable membrane section 15, a substance produced by the cultured organism is allowed to pass through the channel 3 to the outside of the culture tube 2. Accordingly, in each semipermeable membrane section 15, the substance produced by the cultured organism permeates the soil 5, that is the medium, from the channel 3 (arrow B). It is noteworthy that the semipermeable membrane sections 15 allow passage of the produced substance but not cells and the like of the cultured organism to the outside of the culture tube 2. In other words, the semipermeable membrane sections 15 allow passage of the substance produced by the cultured organism to the outside of the culture tube 2 (that is an inside of the soil 5) without allowing passage of the cells of the cultured organism. Herein, examples of the substance produced by the cultured organism includes metabolites, proteins (enzymes) and etc. produced by the cultured organism. The substance may be produced outside the cells of the cultured organism or may be produced inside the cells of the cultured organism.

In the example illustrated in in FIG. 1, a flow rate adjuster 16 is disposed in a branching point of the extensions 11 and 12 of the culture tube 2. The flow rate adjuster 16 includes, for example, a flow rate adjusting valve. In the culture tube 2, a flow rate of the culture solution in the extension (first extension) 11 changes depending on actuation states of the flow rate adjuster 16. Therefore, an amount of the culture solution flowing in from the branching point to each of the extensions 11 and 12 changes depending on actuation states of the flow rate adjuster 16.

The adding device 1 also includes a controller 20. The controller 20 includes a processor or an integrated circuit, and a storage medium such as a memory. The controller 20 controls the actuation of the flow rate adjuster 16. The controller 20 controls the actuation of the flow rate adjuster 16 based on, for example, an operation of a user interface (not illustrated) such as a remote controller. Controlling the actuation of the flow rate adjuster 16 by the controller 20 enables adjustment of amount of the culture solution flowing in from the branching point to each of the extensions 11 and 12, thereby adjusting the flow rate of the culture solution in the extension 11. Furthermore, adjusting the flow rate of the culture solution enables adjustment of amount of the produced substance passing through the semipermeable membrane sections 15 in the extension 11, thereby adjusting an amount of the produced substance added to the soil 5, that is the medium, from the channel 3.

In the example illustrated in FIG. 1, the adding device 1 includes a semipermeable membrane adjuster 18. In one example, the semipermeable membrane adjuster 18 is a drive member such as a motor. The drive of the semipermeable membrane adjuster 18 causes a force transmitted from the semipermeable membrane adjuster 18 to act on the extension 11 where the semipermeable membrane sections 15 are formed and changes the elongation and contraction state of the extension 11. Changing the elongation and contraction state of the extension 11 changes at least one of the interval between the semipermeable membrane sections 15 and a contact area between the semipermeable membrane sections 15 and the soil 5, that is the medium, within the extension 11 of the culture tube 2.

The controller 20 controls the drive of the semipermeable membrane adjuster 18 based on, for example, an operation of a user interface (not illustrated) such as a remote controller. Controlling the drive of the semipermeable membrane adjuster 18 by the controller 20 enables adjustment of the force acting on the extension 11, thereby adjusting elongation and contraction state of the extension 11. Accordingly, in the extension 11 of the culture tube 2, at least one of the interval between the semipermeable membrane sections 15 and the contact area between the semipermeable membrane sections 15 and the soil 5, that is the medium, is adjusted. Adjusting at least one of the interval between the semipermeable membrane sections 15 and the contact area between the semipermeable membrane sections 15 and the soil 5, that is the medium, enables adjustment of amount of the produced substance passing through the semipermeable membrane sections 15 in the extension 11, thereby adjusting an amount of the produced substance added to the soil 5, that is the medium, from the channel 3.

A configuration for adjusting at least one of the interval between the semipermeable membrane sections 15 and the contact area between the semipermeable membrane sections 15 and the soil 5, that is the medium, is not limited to the aforementioned configuration. In one example, the elongation and contraction state of the extension 11 is changed depending on temperature. In this case, adjusting the temperature of the extension 11 enables adjustment of elongation and contraction state of the extension 11 and adjustment of at least one of the interval between the semipermeable membrane sections 15 and the contact area between the semipermeable membrane sections 15 and the soil 5, that is the medium.

It is not always necessary to adjust the amount of the produced substance added to the soil 5 from the channel 3 with the flow rate adjuster 16, the semipermeable membrane adjuster 18, and the like. However, even in a configuration that does not adjust the amount of the produced substance added to the soil 5 from the channel 3, the extension 11 of the culture tube 2 extends inside the soil 5, that is the medium. Furthermore, the semipermeable membrane sections 15 are formed in the extension 11, and the semipermeable membrane sections 15 allow the substance produced by the cultured organism but not the cells of the cultured organism to pass therethrough to the outside of the culture tube 2.

As described above, in the embodiment and the like, the semipermeable membrane sections 15 are formed in a portion of the culture tube 2 extending inside the soil 5, that is the medium. The semipermeable membrane sections 15 allow the substance produced by the cultured organism but not the cells of the cultured organism to pass therethrough, thereby allowing the produced substance to permeate the soil 5 from the channel 3. Accordingly, in the embodiment and the like, it is possible to add the substance produced by the cultured organism to the medium such as the soil 5 while flowing the culture solution containing the cultured organism through the channel 3 inside the culture tube 2. Therefore, it is possible to add the substance produced by the cultured organism to the medium without stopping culture of the cultured organism, that is, while continuing culture of the cultured organism.

For example, assume that a cultured organism is cultured to produce a useful substance such as oil. In this case, adding the substance produced by the cultured organism to a medium while continuing culture concurrently enables production of the useful substance and addition of the produced substance to the medium such as the soil 5. Therefore, it is possible to achieve both the production of the useful substance by culture of the cultured organism and growth promotion of a plant such as a crop by adding the produced substance to the medium. Furthermore, for example, it is possible to achieve both the production of the useful substance by culture of the cultured organism and improvement of crop productivity by adding the produced substance to the medium.

Assume that algae are cultured to produce algae as food. In this case, adding a substance produced by the algae to a medium while continuing culture of the algae concurrently enables production of the algae as food and addition of the produced substance to the medium such as the soil 5. Therefore, it is possible to achieve both the production of the algae as food by culture and growth promotion of a plant such as a crop by adding the produced substance to the medium.

Furthermore, from a viewpoint of carbon dioxide fixation, adding the substance produced by the algae to the medium while continuing culture of the algae enables carbon dioxide fixation by the cultured algae and also increases an amount of carbon dioxide fixed in the plant which has been promoted to grow by the produced substance. Accordingly, it is possible to appropriately achieve an increase in total amount of carbon dioxide fixed in the algae and plant.

In the embodiment and the like, a substance produced by a cultured organism is added to a medium as described above, and there is no need to separate a culture solution that contains the cultured organism into cells of the cultured organism and the produced substance with a centrifuge or the like. Adding the produced substance to the medium without using the centrifuge or the like saves labor during the operation of adding the produced substance to the medium and saves energy for actuating the centrifuge. Further, the cells of the cultured organism not added to the medium can be effectively utilized without being discarded.

In the embodiment and the like, a substance produced by a cultured organism is added to a medium as described above, and there is no need to spread a mixture of cells of the cultured organism and the produced substance on the medium such as the soil 5. Not spreading the cells of the cultured organism appropriately reduces an influence on an environmental ecosystem caused by adding the produced substance to the medium. Further, since the cells of the cultured organism are not spread, a genetically modified cultured organism can be employed.

Furthermore, in one example of the embodiment, the culture tube 2 has the extension (first extension) 11 extending inside the medium (the soil 5), and the semipermeable membrane sections 15 are formed in the extension 11. The culture tube 2 also has the extension (second extension) 12 provided with no semipermeable membrane sections 15 and extending in parallel with the extension 11. Furthermore, the flow rate adjuster 16 is used to adjust the flow rate of the culture solution in the extension 11. Therefore, the flow rate of the culture solution in the extension 11 is adjusted depending on the situation, which appropriately adjusts the amount of the produced substance added to the medium such as the soil 5.

Still further, in one example of the embodiment, at least one of the interval between the semipermeable membrane sections 15 in the culture tube 2 and the contact area between the semipermeable membrane sections 15 and the medium is adjusted with the semipermeable membrane adjuster 18. Therefore, at least one of the interval between the semipermeable membrane sections 15 in the culture tube 2 and the contact area between the semipermeable membrane sections 15 and the medium is adjusted depending on the situation, which appropriately adjusts the amount of the produced substance added to the medium such as the soil 5.

Note that the present invention is not limited to the above embodiments, and various modifications can be implemented without departing from the gist of the invention. Furthermore, the embodiments may be implemented in appropriate combination, and in that case, a combined effect can be obtained. Still further, the embodiments include various inventions, and various inventions can be extracted by a combination selected from a plurality of disclosed components. For example, even if some components are deleted from all the components described in the embodiments, a configuration from which the components have been deleted can be extracted as an invention as long as the problem can be solved and the effects can be achieved.

### REFERENCE SIGNS LIST

1 adding device
2 culture tube
3 channel
5 soil (medium)
6 plant
11 extension (first extension)
12 extension (second extension)
15 semipermeable membrane section
16 flow rate adjuster
18 semipermeable membrane adjuster
20 controller

1. An adding device of a produced substance by a cultured organism, the adding device comprising:
a culture tube that extends into a medium and has a channel through which a culture solution containing the cultured organism flows; and
a semipermeable membrane section that is formed in a portion of the culture tube extending inside the medium and allows a substance produced by the cultured organism to permeate the medium from the channel by allowing the substance but not a cell of the cultured organism to pass through the semipermeable membrane section.

2. The adding device according to claim 1, further comprising:
a first extension of the culture tube extending inside the medium and provided with the semipermeable membrane section;
a second extension extending in parallel with the first extension of the culture tube and not provided with the semipermeable membrane section; and
a flow rate adjuster configured to adjust a flow rate of the culture solution in the first extension to adjust an amount of the substance added to the medium.

3. The adding device according to claim 1, further comprising a semipermeable membrane adjuster configured to adjust at least one of an interval of the semipermeable membrane section in the culture tube and a contact area between the semipermeable membrane section and the medium to adjust an amount of the substance added to the medium.

4. An adding method of a produced substance by a cultured organism, the adding method comprising:
flowing a culture solution containing the cultured organism through a channel inside a culture tube that extends into a medium; and
causing a semipermeable membrane section formed in a portion of the culture tube extending inside the medium to allow a substance produced by the cultured organism to permeate the medium from the channel by allowing the substance but not a cell of the cultured organism to pass through the semipermeable membrane section.

5. The adding method according to claim 4, further comprising adjusting an amount of the substance added to the medium by adjusting a flow rate of the culture solution in a first extension of the culture tube, the culture tube including the first extension extending inside the medium and provided with the semipermeable membrane section and a second extension extending in parallel with the first extension and not provided with the semipermeable membrane section.

6. The adding method according to claim 4, further comprising adjusting at least one of an interval of the semipermeable membrane section in the culture tube and a contact area between the semipermeable membrane section and the medium to adjust an amount of the substance added to the medium.
